(19) United States
(12) Patent Application Publication
(10) Pub. No.: US 2012/0302951 A1
(43) Pub. Date: Nov. 29, 2012

(54) METHOD AND APPARATUS FOR A PRESSURE RELEASE MECHANISM FOR A CATHETER

(75) Inventor: Morgan D'AMORE, Seymour, CT (US)

(73) Assignee: TRAVIS GROUP, LLC, WILMINGTON, DE (US)

(21) Appl. No.: 13/116,160

(22) Filed: May 26, 2011

(51) Int. Cl. A61M 25/10; B23P 19/04

(52) U.S. Cl. 604/96.01; 29/402.03

(57) ABSTRACT

The invention is drawn to a method and apparatus for providing a pressure relief system for a catheter in the form of a fitting, which, when incorporated into the tubing comprising the inflation lumen, will hold the tubing in place while the pressure is below a predetermined level, but will slide out of the tubing, allowing the tubing to separate and releasing the pressure, provided the pressure exceeds that level. Once the pressure has been released, the fitting may be reattached, the conditions which caused the unsafe overpressure may be corrected, and the procedure continued without removal or replacement of the catheter or any of its components.

### FIELD OF THE INVENTION

The present invention relates to a reattachable pressure relief fitting for a catheter. More particularly, the invention comprises a tapered rigid tube adapted to fit firmly between two segments of an inflation lumen, holding the segments of the lumen together under use, but allowing the smaller end of the fitting to slide out of the lumen under overpressure conditions and release the inflation fluid from the catheter. The fitting is reusable once the overpressure has been released by the fitting slipping out of the lumen, wherein it may be reinserted into the lumen and the inflation lumen refilled with fluid.

### BACKGROUND OF THE INVENTION

A number of balloon catheters exist in the prior art. These devices often consist of a shaft which can be inserted into the body, for example through a body orifice or through an incision; can be passed through channels such as blood vessels, the urethra, or various parts of the digestive or respiratory tract; and have a portion designed to expand when an appropriate fluid is added under pressure. An inflation lumen typically runs through the shaft of the catheter from the inflatable portion to an inflating port capped by a one-way valve in the portion of the catheter remaining outside the body. The inflatable portion of the catheter can be inflated by introducing a fluid through the valve and inflation lumen into the inflatable portion, where the increase in the interior pressure causes that portion to expand.

This expansion can be useful for therapeutic purposes, for instance in balloon angioplasty to dilate narrowed blood vessels and crush partially occluding fat deposits therein. The expansion can also be used for mechanical purposes to hold the catheter in place so that the catheter can function either as a drainage tube to remove fluids from inside body cavities, or as a guide for the insertion of surgical instruments. Most catheters have multiple ports and a shaft with multiple lumens so that several functions can be carried out simultaneously or in sequence without removing the catheter.

The fluid used to expand the catheter can be a liquid or a gas, and fluids which will cause minimal damage to the body, such as sterile water, saline solution, air, or carbon dioxide are usually preferred on the chance that rupture of the balloon or other accident will cause release of the fluid into the body.

There are instances, however, when the inflation of the inflatable portion of the catheter can be a hazard. If the pressure that the inflatable portion generates on the surrounding tissue is greater than the pressure that the surrounding tissue can accommodate, inflation of the catheter may tear the surrounding tissue, risking infection and causing damage which may require extensive surgery to correct. A device is desirable, therefore, which would respond to the pressure generated by the inflatable portion of the catheter by releasing that pressure before it exceeds the pressure that the surrounding tissue can accommodate.

One example of a catheter is the Foley catheter, used to drain urine from patients otherwise unable to urinate normally. These catheters consist of a long flexible shaft enclosing multiple lumens, each opening through a port in the portion of the catheter which remains outside the body. The distal end of the catheter is typically inserted into the urethral opening and the catheter is threaded through the urethra and into the bladder. A portion of the distal end of the catheter is inflatable. Once that portion of the catheter has passed through the urethra and into the cavity of the bladder, fluid can be injected through a one-way valve into the inflation lumen, and as the pressure rises, the inflatable portion expands to a size larger than the urethra, preventing the catheter from accidentally sliding or being pulled back out of the body. Typically an opening in the distal tip of the catheter allows urine to drain through a drainage lumen and out a drainage port, and another port leading through a lumen to another opening in the distal tip may be used either to irrigate the bladder or to insert drugs or medications directly into the bladder.

One difficulty of using this type of catheter is determining whether the distal end of the catheter has been passed far enough through the urethra that the entire inflatable portion of the catheter is inside the cavity of the bladder. Inflating that portion while it is still within or partially within the urethra can generate pressures larger than the walls of the urethra can contain, and cause rupture to those tissues, resulting in the necessity for emergency surgery to repair the damage and possible loss of function.

Another difficulty of using these catheters is that they are typically designed for relatively long-term residence within the body, and confused or semi-conscious patients may pull hard enough on the exposed exterior portions of the catheter to pull the inflated portion into the urethra, possibly rupturing the walls of the urethra or the bladder. In addition, the exposed portions of the catheter can catch or hang when the patient is being moved or transported, pulling the inflated portion of the catheter into the urethra and damaging the tissue. All of these forms of damage may be prevented by a device that will release the pressure inside the catheter if it rises beyond a safe level, whether the pressure is caused by attempting to fill the catheter while it is still confined by the urethra or by any external force which might pull the inflated portion into the urethra.

Several devices have been proposed to release this pressure and deflate the catheter before it can cause damage to the surrounding tissue. Some examples follow:

In U.S. Pat. No. 6,375,637, Campbell et al. appeared to propose the inflatable portion of the catheter be provided with a slit or opening, normally covered by an elastic or tape band. Excess pressure inside the inflatable portion of the catheter usually causes a portion of the opening to pull out of the elastic or tape band, releasing the fluid into the body cavity at the distal end of the catheter. Although this typically releases the pressure, it may not be an optimal solution because the fluid is released into the body, and. the catheter must be completely removed from the body and reassembled before its use can be continued.

Newcomb, in US 2006/0161102, and Pepper, in US 2010/0217189, seem to describe pressure release systems for catheters used in blood vessels. These catheters normally include a relief valve in the form of a thin membrane which ruptures when the pressure exceeds a predetermined limit, releasing inflating fluid and reducing the pressure. Although these catheters may include provisions for mounting the relief valve outside the body and thus releasing the inflating fluid outside the body in the event of overpressure, the continued use of this form of catheter often requires the installation of a new membrane, which may require removal of the catheter from the body.

Kaiser, in US 2006/0167438, generally describes another type of pressure release system in which the inflation lumen of the catheter is manufactured with a weak spot, designed to rupture before the pressure within the inflatable portion exceeds the tolerated. pressure of the surrounding tissue. In this system, the entire catheter usually must be removed and replaced to continue the procedure. A second system described in Kaiser usually has the pressure relief valve mounted on a removable hub exterior to the body, but again the valve normally must be replaced in the event of overpressure.

All of these known methods of releasing pressure from a balloon catheter involve complications in the manufacture of the catheter to produce weak spots with the appropriate bursting characteristics, and require replacement of either the burst membrane or the entire catheter in the event of a pressure release.

What is desired, therefore, is a pressure relief system for a catheter that would release pressure reliably before it can exceed the tissue strength of its surroundings, and that would not require additional complications in the manufacture of the catheter. Another desire is a pressure relief system for a catheter that could be easily reset and reused without removing the catheter or replacing any components thereof. A further desire is a pressure relief system for a catheter that can be easily adapted to existing catheters.

### SUMMARY OF THE INVENTION

It is therefore an object of this invention to provide a pressure relief system for a catheter in the form of a fitting, which, when incorporated into the tubing comprising the inflation lumen, will hold the tubing in place while the pressure is below a predetermined level, but will slide out of the tubing, allowing the tubing to separate and releasing the pressure, provided the pressure exceeds that level. Once the pressure has been released, the fitting may be reattached, the conditions which caused the unsafe overpressure may be corrected, and the procedure continued without removal or replacement of the catheter or any of its components.

A further object of this invention is to provide a catheter incorporating such a pressure relief system, to prevent unsafe overpressure conditions from damaging the internal structure of the body.

Another object of this invention is to provide a method for using a catheter incorporating such a pressure relief system, enabling overpressure to be released by the separation of the fitting from the inflation lumen, but allowing the procedure to continue without removal or replacement of the catheter or any of its components by reattaching the pressure relief fitting.

Yet another object of this invention is to provide a method for incorporating such a pressure relief system into existing catheters.

These and other objects of the invention are achieved by providing a pressure relief system incorporating a fitting which can be used to releasably and reattachably connect two segments of tubing, particularly by releasing in response to a rise in pressure above a predetermined level.

In some embodiments, the fitting may have a smaller diameter first end and a larger diameter second end. In some embodiments, the larger diameter of the fitting is slightly larger than the resting inside diameter of a segment of tubing, such that the tubing must be stretched slightly to go over the end of the fitting, holding it tightly in place. In some embodiments, the smaller diameter is slightly smaller than the resting inside diameter of a segment of tubing, such that it will slide easily into the tubing, stretching the tubing as the diameter of the fitting increases, but allowing it to slide back off if the pressure inside the tube increases beyond a predetermined level. In some embodiments, the pressure required to release the fitting may be determined in part by the depth to which the smaller diameter end of the fitting is inserted into the flexible tubing.

Another embodiment of this invention is a pressure relief system comprising a catheter incorporating a reattachable pressure relief fitting. In some embodiments, the pressure relief fitting may be attached to, or manufactured as part of, the end fitting on the inflation lumen. In other embodiments, the lumen may be separated into two segments, each fitted to one end of the pressure relief fitting. In some embodiments, an increase in pressure to a predetermined level inside the inflation will cause the fitting to detach from the lumen and release the pressure.

Another embodiment of this invention is a method of adapting an existing catheter to incorporate a pressure relief fitting, comprising separating a portion of the inflation lumen of the catheter into two segments, inserting the larger diameter end of the fitting into one segment, and inserting the smaller diameter end of the fitting into the other segment, connecting the segments of the lumen. In this embodiment, an increase in pressure to a predetermined level in the inflation lumen of the modified catheter will cause the pressure relief fitting to release from the lumen.

Another embodiment of this invention is a method of using a catheter incorporating a pressure relief system wherein the catheter is inserted into a body cavity or body part, an inflatable portion of the catheter is inflated, and if the pressure rises beyond a predetermined level, the fitting slides out of the inflation lumen, releasing the pressure. In some embodiments, the fitting may be reattached to the inflation lumen without the necessity of removing the catheter or replacing any of its parts, and the catheter may be reinflated, permitting the procedure to continue.

### BRIEF DESCRIPTION OF THE DRAWINGS

The accompanying drawings show only typical embodiments of the present invention and are not intended to be limiting of its scope.

FIG. 1 shows a side view of the pressure release system in accordance with the invention.

FIG. 2 shows an end view of the pressure release system shown in FIG. 1.

FIG. 3 shows an assembly view of the pressure relief system shown in FIG. 1.

FIG. 4 shows a method of providing the pressure relief system shown in FIG. 1.

FIGS. 5a, 5b, and 5c depict various points along the method shown in FIG. 3.

### DETAILED DESCRIPTION

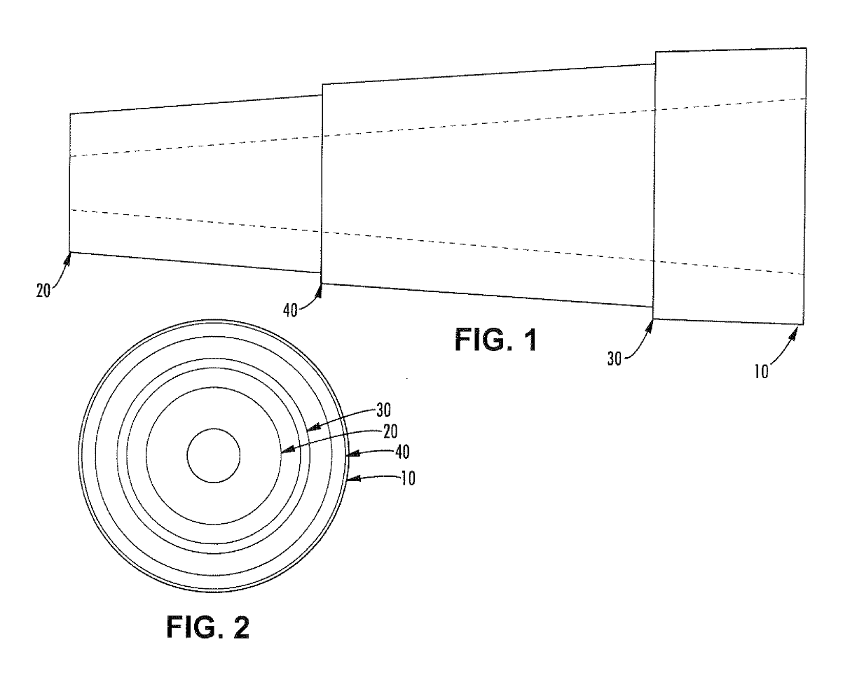

FIG. 1 depicts one embodiment of the invention, a fitting in the form of a tapered rigid tube to be inserted between two segments of tubing. The first end of the fitting (10) is manufactured slightly larger than the interior diameter of the tubing. One segment of tubing is stretched to fit over the larger end of the fitting, holding it firmly. The second end of the fitting (20) is inserted into the second segment of tubing to a predetermined depth, the taper of the fitting stretching the second segment of tubing. The fitting will now act as a pressure relief system sensitive to pressures inside the tubing. As the pressure increases inside the tubing, force will be generated tending to push the second segment of tubing off the fitting, that force counteracted by the friction between the fitting and the inside of the tubing. When the force generated by the pressure inside the tubing exceeds the force due to the friction between the tubing and the fitting, the tubing will slip off the fitting and release the pressure. The deeper the fitting is inserted into the second segment of tubing, the higher the friction will be between the fitting and the tubing, and the higher the pressure that will be needed to release the fitting.

The inside of the fitting is hollow so that fluid will flow easily through the fitting from one segment of tubing to the other. The type of fluid used will depend on the application and can include gases, liquids, or fluidized particles.

In some embodiments, the outside surface of the fitting may be tapered from the larger diameter end to the smaller diameter end, forming a truncated conical shape. In other embodiments, the taper of the tubing may change in a series of steps of abruptly increasing or decreasing sizes. In other embodiments, the taper of the fitting may follow other contours, such as hyperbolic or parabolic curves, or bulbous centers with tapered ends. In some embodiments the outer surface of the fitting may be textured or knurled to increase the amount of pressure required to release the fitting from the tubing, or may be lubricated or polished to decrease the amount of pressure required to release the fitting.

The outside of the fitting may be further modified. to increase or decrease the amount of force required to release the fitting for the. In the embodiment shown in FIG. 1, the outside taper has been formed with two steps (30, 40), or relatively abrupt changes in diameter. One segment of tubing is stretched to fit over the larger end of the fitting (10), which is inserted such the end of the tubing extends beyond the first step (30). This allows the end of the tubing to contract, closing over the edge of the first step and making it more difficult for the larger end of the fitting to slide out of the tubing. The smaller end of the fitting (20) is inserted into a second segment of tubing to the depth indicated by the second step (40). Since the friction between the fitting and the second segment of tubing is determined partially by the depth to which the fitting is inserted into the tubing, inserting the fitting to the depth of the step (40) allows the pressure required to release the fitting to be reliably reproduced. The level of pressure at which the fitting will release can thus be determined by selecting the shape and surface characteristics of the fitting and controlling the depth to which the fitting is inserted into the tubing.

Other external configurations of the fitting can be envisioned for other applications. For example, the center section could be made larger than either of the two end sections to make it easier to grip while inserting the fitting into the tubing, and a variety of ergonomic forms can be envisioned to further improve ease of manipulation. The center could be square or hexagonal or of some other shape, to permit it to be fitted or clamped into an external holder. The center section could alternatively be made smaller than either of the two ends, to fit other types of clamps or to allow it to be strapped into place while maintaining a low profile.

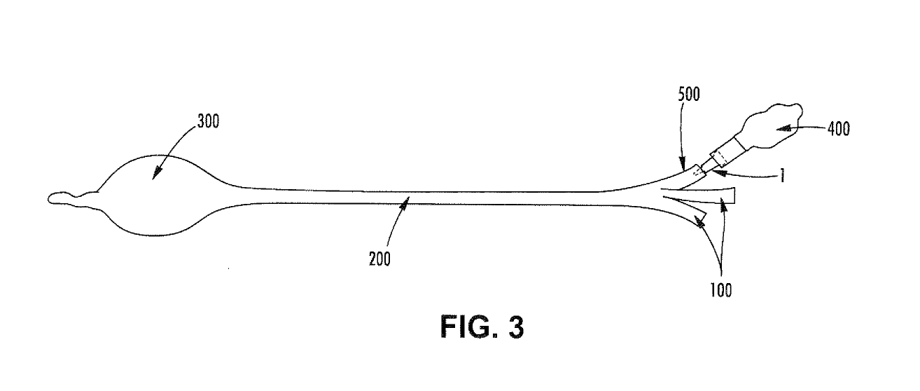

The embodiment shown in FIG. 1 and FIG. 2 is especially adapted for use in a modified Foley catheter. A pressure relief system exemplifying this embodiment is shown in FIG. 3. This figure depicts a modified Foley catheter having three lumens which are separate sections of tubing (100, 500) on the proximal portion of the catheter (that part located outside the patient when deployed), and converge into separate ducts running through the shaft (200) that makes up the body of the catheter. A segment (300) of the distal portion of the catheter (that part located inside the patient when deployed) is designed to expand when inflated by a fluid injected or pumped through one of the lumens, the inflation lumen (500). The proximal end of the inflation lumen is typically capped with a valve (400) to hold the fluid inside the catheter. Typically, this valve is a one-way valve which will allow fluid to pass into the inflation lumen and hold the pressure inside the catheter until it is manually released. The modification involves the incorporation of a pressure relief fitting (1), located in the inflation lumen between the one-way valve (400) and the shaft (200).

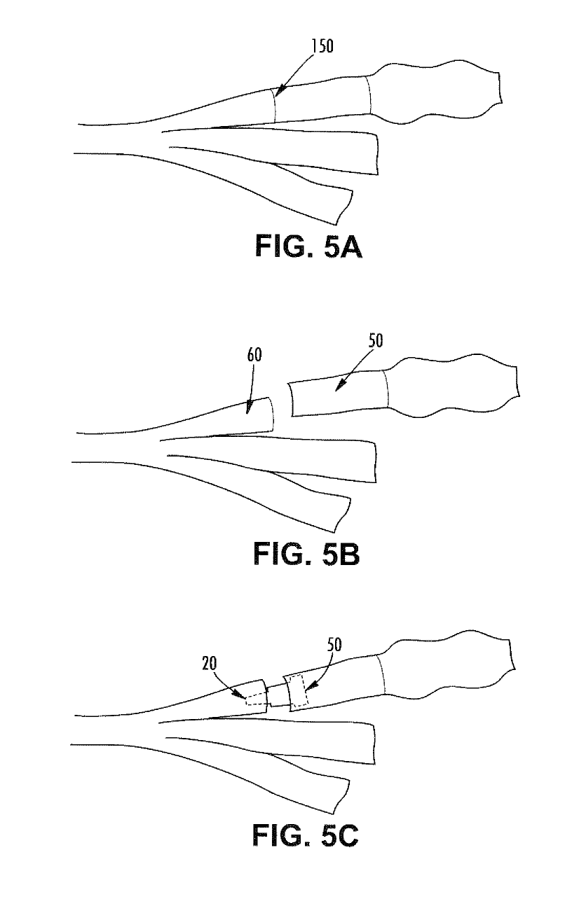

To adapt a catheter to employ this embodiment of the invention, the inflation lumen may be separated into two segments at a point (150) between the proximal end and the entry into the shaft of the catheter as shown in FIGS. 5a and 5b. The fitting is inserted between the two segments of the inflation lumen, the larger end (10) fitted into the proximal segment of tubing (50) and the smaller end (20) fitted into the distal segment (60), as shown in FIG. 5c.

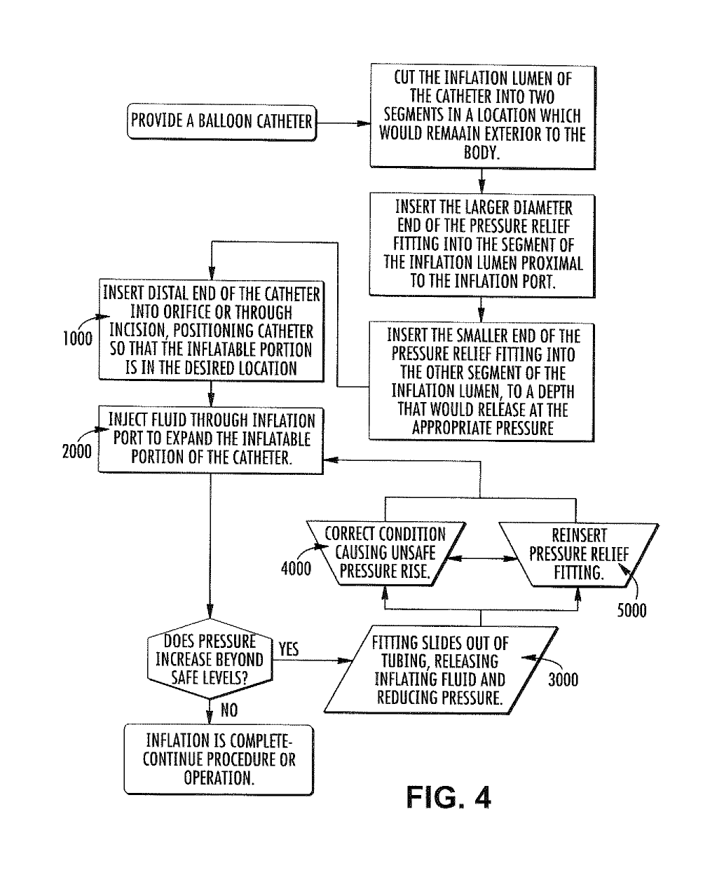

The catheter may now be used as shown in the flowchart in FIG. 4, with the additional safety provided by the pressure relief fitting. The catheter is inserted into the opening of the urethra and threaded through the urethra until the inflatable portion of the catheter is thought to be inside the bladder (1000). Fluid is pumped or injected through the one-way valve (400) and into the inflation lumen of the catheter (2000). If the catheter is not properly located and the inflatable portion is still partially or wholly within the urethra, attempted inflation will cause the pressure to rise above the predetermined safe level and the fitting will detach from the tubing (3000), releasing the contained fluid and the pressure. The fitting can be reattached (4000), the catheter repositioned (5000), and the inflation process repeated.

Once the catheter is located in the correct position and inflated properly, the pressure relief system also prevents damage to the urethra in response to sudden pulls or tugs on the proximal end of the catheter. This can be a problem especially with confused or semi-conscious patients. If a pulling force is applied to the proximal end of a catheter with no pressure relief system, it tends to pull the inflated portion of the catheter into the distal opening of the urethra, exerting pressure on the urethra and if enough force is applied, tearing the wall of the urethra or bladder. With the pressure relief fitting of this invention in place, before the catheter can damage the urethra, the pressure inside the inflatable portion of the catheter will have risen high enough to detach the fitting from the inflation lumen, releasing the fluid and the pressure without damaging the wall of the urethra. Once the source of the problem has been corrected, the fitting may be reattached, the catheter repositioned if needed, and the inflatable portion reinflated. Since the pressure relief system is located outside of the body in this embodiment, the catheter will normally not need to be removed from the body under these circumstances. Since the fitting is reattachable, no pieces will normally need to be replaced before it is repositioned and reinflated.

The pressure relief fitting need not be inserted between two segments of tubing, but may be manufactured as a portion of an end fitting to the tubing, for example, as a part of the one-way valve typically found terminating the inflation lumen of a catheter. In this embodiment, the first end of the fitting may be attached directly to the one-way valve, either as an extension of the body of the valve during manufacture, or by attachment means, for example complimentary threads manufactured into the valve and the fitting, adhesives used to attach the valve body to the fitting, solvent welding, or heat welding. In applications which do not have a one-way valve, the second end of the pressure relief fitting may be inserted into the tubing and the first end attached directly to whatever means are used to insert fluid into the tubing, such as a syringe adapter or tubing or pipe connector. In other embodiments, the first end may be capped or plugged and the second end inserted into a tubing to act as a terminus, which will release if the pressure inside the tubing rises above a predetermined level. Other means of attaching components of a fluid-handling system to one end of the fitting while leaving the other end free to release from the tubing to release pressure may be envisioned and are intended to be included within the scope of this invention.

This pressure relief system may be adapted to other fluid handling applications, especially those which involve inflation. Tubing carrying compressed air for inflating tires, balloons, or sports equipment may be equipped with a similar pressure relief fitting, to prevent overinflation or excessive pressure from damaging the inflated items. In this embodiment, the tubing may be separated at any point between the pump or other source of compressed air and the object being inflated, and a pressure relief fitting used to reconnect the tubing. Fill tubes for water-beds, air mattresses, or compressed gas tanks may be so equipped to prevent bursting either of the object being filled or the fill tubing. Other applications might include hydraulics systems, in which a pressure relief fitting of this type would release the hydraulic fluid in the event of pressures which would damage either the hydraulic system or the objects on which it is acting.

What is claimed is:

1. A pressure relief system comprising a fitting having a smaller diameter first end and a larger diameter second end, said first end releasably fits into a first tubing and said second end releasably fits into a second tubing, said tube adapted to reattachably release from either said first or second tubing.

2. A pressure relief system as described in claim 1 wherein a rise in pressure inside said first or second tubing causes said fitting to reattachably release^{.} from said first or second tubing.

3. A pressure relief system as described in claim 1 wherein a rise in pressure above a predetermined level causes said fitting to reattachably release from said first or second tubing.

4. A pressure relief system, comprising:
a catheter having a lumen; and
a fitting having a first end, such that said first end fits into said lumen, said fitting adapted to reattachably release from said lumen.

5. A pressure relief system as described in claim 4, wherein said lumen comprises a first and second tubing, the first end of said fitting releasably fits into said first tubing, and the second end of said fitting releasably fits into said second tubing.

6. A pressure relief system as described in claim 4, wherein said fitting is adapted to reattachably release from said lumen if a pressure inside said lumen rises above a predetermined level.

7. A method of providing a pressure relief system comprising the steps of:
providing a catheter having a lumen;
separating the lumen into a first and second tubing;
providing a fitting having a smaller diameter first end and a larger diameter second end; and
inserting the smaller diameter first end inside the first tubing and inserting the larger diameter second end inside the second tubing.

8. The method of providing a pressure relief system as described in claim 7, further comprising the steps of:
providing the first tubing with an inner diameter large enough for placing the first end into the inner diameter of the first tubing, wherein the fitting is releasably attached to the first tubing;
providing the second tubing with an inner diameter large enough for placing the second end into the inner diameter of the second tubing, wherein the fitting is releasably attached to the second tubing.

9. The method of using a pressure relief system as described in claim 7, further comprising the steps of:
inflating a portion of said catheter;
avoiding a pressure above a predetermined level by said fitting releasing from said lumen when the pressure rises above a predetermined level.

10. The method described in claim 9, further comprising the steps of:
reinserting said fitting into said lumen; and
reinflating a portion of said catheter.
